(19) United States
(12) Patent Application Publication
(10) Pub. No.: US 2016/0266019 A1
(43) Pub. Date: Sep. 15, 2016

(54) METHOD FOR SEPARATING MULTIPLE BIOLOGICAL MATERIALS

(71) Applicant: University-Industry Foundation, Yonsei University, Seoul (KR)

(72) Inventors: Seungjoo HAAM, Seoul (KR); Yong-min Huh, Seoul (KR); Byunghoon Kang, Seoul (KR); Eunji Jang, Seoul (KR); Seungmin Han, Seoul (KR); Hyun-Ouk Kim, Seoul (KR)

(21) Appl. No.: 15/064,630

(22) Filed: Mar. 9, 2016

Related U.S. Application Data
(63) Continuation-in-part of application No. PCT/KR2014/008102, filed on Aug. 29, 2014.

(30) Foreign Application Priority Data
Aug. 25, 2014 (KR) 10-2014-0110777
Aug. 25, 2015 (KR) 10-2015-0119330

(51) Int. Cl. G01N 1/40; G01N 33/543; B01L 3/00; G01N 33/553

(52) U.S. Cl. CPC G01N 1/405; G01N 33/553; G01N 33/54333; G01N 33/54346; G01N 33/54366; B01L 3/502715; B01L 3/502761; G01N 2001/4038; B01L 2200/0668; B01L 2300/06; B01L 2200/0631; B01L 2300/12

(57) ABSTRACT

The present invention relates to a method for separating multiple biological materials using characteristics of magnetic nanoparticles. The method of the present invention is able to separate multiple biological materials at once due to differences in trajectory in an external magnetic field of the same intensity after attaching magnetic nanoparticles to the biological materials to be separated using differences in magnetic susceptibility or magnetization depending on compositions of the magnetic nanoparticles.

### RELATED APPLICATIONS

This application is a Continuation-in-Part (CIP) of PCT Patent Application No. PCT/KR2014/008102 having International filing date of Aug. 29, 2014, which claims the benefit of priority of Korean Patent Application No. 10-2014-0110777 filed on Aug. 25, 2014.

This application also claims the benefit of priority of Korean Patent Application No. 10-2015-0119330 filed on Aug. 25, 2015.

The contents of the above applications are all incorporated by reference as if fully set forth herein in their entirety.

### FIELD AND BACKGROUND OF THE INVENTION

The present invention relates to a method for separating of biological materials using characteristics of a magnetic nanoparticle.

Separation of a cellular type or intracellular components is required as a preparation tool for diagnosis and treatment in medical and pharmaceutical fields and for achieving a final objective or performing another analysis in a research field. Currently, there are many types of cell sorting methods being used in laboratories and clinical laboratories. Rapidly separating different types of particles, e.g. viruses, bacteria, cells, and multicellular organisms, is a central step in various applications in medical and pharmaceutical research areas, a clinical diagnosis area, and an environmental analysis area. Rapidly growing knowledge in new drug development and protein research is enabling researchers to promptly obtain a better understanding of protein-protein interactions, cell signaling pathways, and markers of metabolic processes. It is difficult or impossible to acquire the above information using a single protein detection method, e.g. conventional methods such as enzyme-linked immunosorbent assay (ELISA) or Western blotting.

Since microfluidics improve regeneration while reducing time and cost related to a conventional analysis, there has recently been an effort to change a conventional biological task to a lab-on-a-chip system. The most important tools that improve the efficiency of characteristic identification and preparation steps of biological materials are an ability to recognize a target component in a mixture and selectively control, interact and/or isolate the target component. Also, a microbead-based analysis has advantages compared to a flat microarray. Cleaning is efficient, multiple analyses are possible using an encrypted microbead, a signal is amplified due to a large surface-to-volume ratio, and an analysis time is short since the microbead may freely move within a media.

Separation performance is evaluated using the following three characteristics. “Throughput” refers to a number of analytes that may be identified and sorted per unit hour, and “purity” refers to a fraction of a target analyte in a trapping region. “Recovery rate” refers to a fraction of an injected target analyte that is successfully sorted into the trapping region. A fluorescence activated cell sorter (FACS), a dielectrophoretically activated cell sorter (DACS), and a magnetically activated cell sorter (MACS) have been used to separate and control cells [Current Opinion in Chemical Engineering, 2013, 2, 3-7]. Although these technologies provide a high specificity in cell separation, there are disadvantages. For example, FACS has a limited throughput (mostly 10³-10⁴ cells/second). Also, the sorting time is long and a mechanical stress of its nozzle is great, thus causing a cell survival rate to decrease and a functional cell survival rate to also decrease. Also, it is costly, and the design and operation thereof are complicated.

In addition, Lab Chip, 2011, 11, 1902-1910 relates to a cell separation method using a microfluidic channel and a magnetic field, and although a technology of separating cells by controlling a loading amount of iron and a flow speed of a magnetic body is disclosed, there is an inconvenience of controlling an intracellular treatment time to control the loading amount of the magnetic body.

### SUMMARY OF THE INVENTION

### Technical Problem

Thus, as a result of studying to solve the problems of cell separation methods using the conventional MACS and the existing microfluidic channel, a method for separating multiple biological materials by changing a composition of a magnetic nanoparticle to control magnetic susceptibility or magnetization has been developed, thus completing the present invention.

Therefore, the present invention is directed to providing a method for separating biological materials by a microfluidic channel using characteristics of a magnetic nanoparticle.

### Technical Solution

One aspect of the present invention provides a method for separating multiple biological materials, the method including separating multiple biological materials using magnetic susceptibility or magnetization of three or more types of magnetic nanoparticles having different compositions which are expressed by Chemical Formula 1 below:

MFe₂O₄ Chemical Formula 1

In Chemical Formula 1, M is Fe, Mn, Co, Ni, or Zn.

Another aspect of the present invention provides a method for separating multiple biological materials, the method including respectively coupling three or more types of magnetic nanoparticles to three or more types of biological materials to be separated in a sample; injecting the sample and a buffer into a microfluidic channel; generating a magnetic field outside of the microfluidic channel while the sample and the buffer are passing through the microfluidic channel; and separating the biological materials to different movement pathways due to differences in magnetic susceptibility or magnetization of the magnetic nanoparticles, wherein the magnetic nanoparticles are expressed by Chemical Formula 1 below:

MFe₂O₄ Chemical Formula 1

In Chemical Formula 1, M is Fe, Mn, Co, Ni, or Zn.

### Advantageous Effects

According to the present invention, a method for separating multiple biological materials is able to separate multiple biological materials at once due to differences in trajectory in an external magnetic field of the same intensity after attaching magnetic nanoparticles to biological materials to be separated using differences in magnetic susceptibility or magnetization depending on compositions of the magnetic nanoparticles. Particularly, since the present invention only has to change compositions of the magnetic nanoparticles such that the magnetic nanoparticles are attached to surfaces of the biological materials, a treatment time can be considerably reduced compared to the cell separation method using the existing microfluidic channel, and the separation is possible even without biological material specificity.

In addition, although approximately 10,000 biological materials can be separated per minute in the case of the conventional separation method being used, approximately 100,000 biological materials can be separated when the separation method of the present invention is used. Furthermore, although an analysis of multiple biological materials and the separation thereof have been performed in separate systems conventionally, the analysis and the separation are possible within one system in the present invention, and the biological materials can be reused as biological samples since their cells can be reacquired after separating the biological material.

### BRIEF DESCRIPTION OF THE SEVERAL VIEWS OF THE DRAWINGS

FIG. 1 illustrates magnetization intensities of magnetic nanoparticles, compositions of which have been controlled, a microfluidic channel structure for separating multiple biological materials using the same, and a principle of multicellular separation depending on the magnetization intensities.

FIG. 2 illustrates the microfluidic channel structure for separating multiple biological materials according to the present invention.

FIGS. 3A-3C illustrate scanning electron microscopy images of magnetic nanoparticles used in biological material separation [A) Fe₃O₄; B) MnFe₂O₄; C) CoFe₂O₄].

FIGS. 4A-4C illustrate results of measuring magnetization of each of the magnetic nanoparticles by a vibrating sample magnetometer (VSM) [A) Fe₃O₄; B) MnFe₂O₄; C) CoFe₂O₄].

FIGS. 5A-5C illustrate electron microscopy images of cells coated with magnetic particles having different compositions [A) a jurkat cell coated with Fe₃O₄; B) a jurkat cell coated with MnFe₂O₄; C) a jurkat cell coated with CoFe₂O₄].

FIGS. 6A-6C illustrate results of measuring magnetic susceptibility after treating the jurkat cells with each of the magnetic nanoparticles by the VSM [magnetic susceptibility of the jurkat cell treated with each of A) Fe₃O₄; B) MnFe₂O₄; C) CoFe₂O₄].

FIGS. 7A-7C illustrate electron microscopy images of cells coated with magnetic particles having different compositions [A) a jurkat cell coated with Fe₃O₄; B) an SK-BR-3 cell coated with MnFe₂O₄; C) an A431 cell coated with CoFe₂O₄].

FIGS. 8A-8C illustrate results of measuring magnetic susceptibility after treating each of three or more types of cells with each of the magnetic nanoparticles by the VSM [magnetic susceptibility of A) the jurkat cell coated with Fe₃O₄; B) the SK-BR-3 cell coated with MnFe₂O₄; C) the A431 cell coated with CoFe₂O₄].

FIGS. 9 and 10 illustrate trajectories resulting from simulating differences in behaviors of cells by controlling the size of an external magnetic field.

FIG. 11 illustrates a fluorescence microscopy image of cellular trajectories in the microfluidic channel after injecting cells treated with each of the magnetic nanoparticles under the external magnetic field of a predetermined size into a sample injection unit and a buffer into a buffer injection unit. Also, results of an elementary analysis of each of the samples from which cells are separated by an inductively coupled ion plasma are shown.

FIG. 12A illustrates results of FACS analysis after performing cellular separation by injecting a mixture of cells treated with each of the magnetic nanoparticles under the external magnetic field of a predetermined size into the sample injection unit and the buffer into the buffer injection unit.

FIG. 12B is a view setting separation sections of the cells in FIG. 12A.

### DESCRIPTION OF SPECIFIC EMBODIMENTS OF THE INVENTION

The present invention relates to a method for separating multiple biological materials, the method including separating multiple biological materials using magnetic susceptibility or magnetization of three or more types of nanoparticles having different compositions which are expressed by Chemical Formula 1.

As one embodiment, the present invention includes a method for separating multiple biological materials, the method including respectively coupling three or more types of magnetic nanoparticles to three or more types of biological materials to be separated in a sample; injecting the sample and a buffer into a microfluidic channel; generating a magnetic field outside of the microfluidic channel while the sample and the buffer are passing through the microfluidic channel; and the biological materials being separated to different movement pathways due to differences in magnetic susceptibility or magnetization of the magnetic nanoparticles.

The biological materials may be viruses, bacteria, cells, intracellular organs, molecules, or multicellular organisms.

The magnetic nanoparticles are preferably expressed by Chemical Formula 1 below:

MFe₂O₄ Chemical Formula 1

In Chemical Formula 1, M is preferably a transition metal that has stronger magnetic characteristics as its atomic number gets smaller and its number of unpaired electrons increases, and is specifically Fe, Mn, Co, Ni, or Zn.

A Bohr magneton changes in accordance with a type of the transition metal doped inside a unit cell of the magnetic nanoparticles and shows a great difference when calculated with respect to the entirety of particles, thus affecting magnetic susceptibility or magnetization. When the same sample is treated with magnetic nanoclusters having different compositions using the difference, the magnetic susceptibility or magnetization changes, thus enabling the separation.

An average size of the magnetic nanoparticles is preferably within a range of 10 to 200 nm, and sizes of the magnetic nanoparticles having different compositions being used are preferably the same such that the intensity of the magnetic susceptibility or magnetization is unaffected. When the size of the particles enlarges, a resolving power decreases due to a precipitation phenomenon caused by gravity even though a difference in the susceptibility becomes greater.

First, various biological materials and the magnetic nanoparticles are coupled to separate the multiple biological materials. Here, an antigen-antibody reaction, a selective coupling reaction using a specific genetic combination (aptamer), coupling using a surface charge, and the like may be used as a method for coupling the biological materials to the magnetic nanoparticles.

Although an injection speed of a sample including the multiple biomaterials to be separated may vary in accordance with the size of the microfluidic channel, being within a range of 1 μl/min to 50 μl/min is preferable for the biological materials treated with the magnetic nanoparticles to be effectively affected by the magnetic field.

In addition, an injection speed of a buffer used for constant laminar flow is preferably 8 μl/min to 400 μl/min according to the injection speed of the sample.

When the magnetic field of a predetermined size is generated outside the microfluidic channel using a presence of differences in the intensity of the magnetic susceptibility or magnetization of the magnetic nanoparticles, movement pathways of the magnetic nanoparticles having different compositions become different due to magnetic characteristics, thus being able to separate various biological materials to which the magnetic nanoparticles having different compositions are coupled at once. Here, the external magnetic field is preferably generated in a direction perpendicular to a fluid flowing direction in the microfluidic channel to maximize differences in the movement pathways for effective separation. Also, the intensity of the magnetic field is preferably in the range of 500 G to 3,000 G (0.05 T to 0.3 T). The separation of the biological materials becomes difficult when the intensity is too weak, and the separation of the biological materials is impossible when the intensity is too strong due to radical changes of the movement pathways.

In addition, as one embodiment, the present invention includes a method for separating multiple biological materials using an apparatus for separating multiple biological materials, the apparatus includes a microfluidic channel structure including an injection unit into which a plurality of samples and a buffer are injected, a main channel in which biological materials are separated by an external magnetic field, and a discharge unit which discharges a plurality of separated biological materials, and a magnetic device to form a magnetic field along one direction different from a fluid flowing direction in the main channel.

The apparatus for separating multiple biological materials that is used in the present invention is formed of the microfluidic channel formed between an upper substrate and a lower substrate and having a sample including a magnetic body and micro-particles passed therethrough, and the magnetic device including an external magnetic field source to generate the magnetic field therearound.

The microfluidic channel may be divided into the injection unit into which the sample including micro-particles and the buffer are injected; the main channel through which biological materials included in the injected sample pass while being separated by the magnetic field; the discharge unit including a plurality of outlets through which each of the biological materials separated while passing the main channel and remaining samples are separated and discharged.

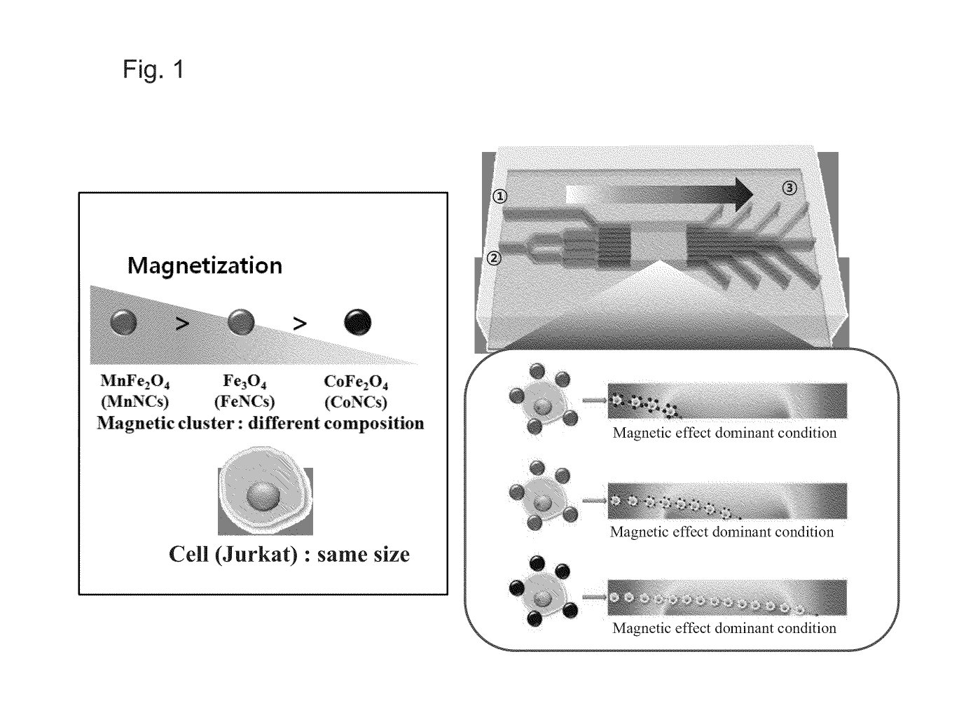

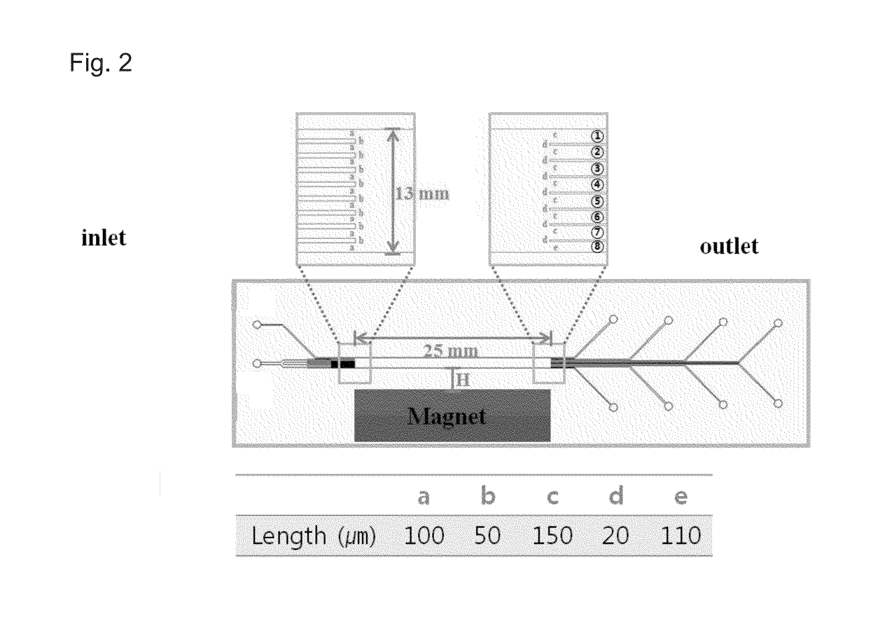

In FIG. 1, the \textcircled{1} portion is a sample injection unit and the \textcircled{2} portion is a buffer injection unit, thus enabling a trajectory of the sample treated with the magnetic nanoparticles to be checked. The \textcircled{3} portion is the discharge unit and enables the sample to be separated according to the trajectory. A reason of performing the sample injection at a wall side is to maximize changes in the trajectory, and a channel shape of the buffer injection unit is manufactured as shown in FIG. 2 in order to maximally suppress the laminar flow and make the speed of a fluid constant.

The speed becomes constant when a fraction \textcircled{2}/\textcircled{1} is 8. Also, when a number of the buffer injection units is increased, the speed may be constant even when the speed is increased corresponding to the fraction, and a buffer effect may be increased to maximize the laminar flow effect.

The sizes of the magnetic nanoparticles are the same to check the differences in susceptibility in accordance with changes in compositions.

The Bohr magneton changes in accordance with a type of the transition metal doped inside a unit cell of the magnetic nanoparticles and shows a great difference when calculated with respect to the entirety of the particles, thus affecting magnetic susceptibility or magnetization. When the same sample is treated with magnetic nanoparticles having different compositions using the difference, the magnetic susceptibility or magnetization changes, thus enabling the separation. Furthermore, when the compositions of the magnetic nanoparticles are the same while the sizes thereof are different, the magnetic susceptibility or magnetization increases as the size increases.

The microfluidic channel structure is manufactured by applying a photolithography process used in semiconductor manufacturing. Various polymer materials such as polydimethyl siloxane (PDMS), polymethyl methacrylate (PMMA), polycarbonate (PC), polyethylene (PE), polypropylene (PP) polystyrene (pS), polyolefin, polyimide, and polyurethane may be used as a material for manufacturing the microfluidic channel structure.

The flows of the sample and the buffer in the microfluidic channel structure may be controlled using methods well-known in the area. Methods using an electroosmotic flow, a membrane pump, and a syringe pump that electrically move a small amount of liquid sample are included in the above methods.

In the embodiment of the present invention, the microfluidic channel structure was manufactured by attaching a patterned PDMS channel to a lower glass substrate.

When the fluid speed in the microfluidic channel is too slow, the biological materials are not separated due to being pulled down by gravity caused by density of cells and not flowing. When the fluid speed therein is too fast, the biological materials are not separated since all of the cells are discharged to the discharge unit before being affected by a force by the magnetic field. The flow speed experiment may be influenced by a correlation between the channel size and the flow speed, and particularly, a fluid gradient of a parabolic form is generated in the flowing fluid, and it is important to design the microfluidic channel in a shape of a plug flow by having a plurality of buffer inlets in order to eliminate the influence of the fluid gradient. Also, a problem of having to consider the separation in terms of the flow at the same time as the actual separation by the magnetic field may be solved by setting an optimal number of buffer inlets.

That is, although it is preferable to maximize the number of buffer inlets among the injection unit of the microfluidic channel by increasing a number of buffer solution channels to reduce the parabolic fluid flow having the laminar flow effect, the number is limited corresponding to the size thereof due to a limitation in manufacturing the microfluidic channel. Consequently, the buffer inlets are preferably formed of 8 to 20 channels. When the number of the channels of the buffer inlets is small, a fluid speed at a central portion in the channel is faster than the fluid speed at the wall.

In addition, the microfluidic channel structure is manufactured to promptly flow a phosphate buffered saline (PBS) solution which is mixed with a predetermined amount of bovine serum albumin (BSA) therethrough after being manufactured in order to reduce a phenomenon in which the biological materials are stuck to surfaces of the microfluidic channel and fluid bubbles form.

In addition, the magnetic device may include applying an external magnetic field from a permanent magnet or an electromagnet.

The permanent magnet may be formed from nickel, cobalt, iron, and alloys thereof and alloys of non-ferromagnetic materials, i.e. alloys known as Heusler alloys (e.g., alloys of copper, tin, and manganese), that become ferromagnetic by being alloyed. Many proper alloys for the permanent magnet are known, and may be commercially used to manufacture a magnet that may be used in the embodiment of the present invention. The typical material is a transition metal-semimetal (metalloid) alloy, which is formed from approximately 80% of a transition metal (usually Fe, Co, or Ni) and a semimetal component (boron, carbon, silicon, phosphorus, or aluminum) that lowers a melting point. The permanent magnet may be crystalline or amorphous. Fe80B20 (Metglas 2605) is an example of an amorphous alloy. The external magnetic field used in the embodiment of the present invention is provided by a rectangular (2.5 cm×2.5 cm×4.0 cm) NdFeB magnet (K&J Magnetics, Jamison, Pa.) attached to an upper surface and a lower surface of the main channel of the microfluidic channel.

Hereinafter, the present invention will be described in more detail by embodiments according to the present invention, but the scope of the present invention is not limited by the embodiments described below.

### EMBODIMENTS

### Example 1

### Fabrication Microfluidic Channel Apparatus

To form the microfluidic channel, a pattern using an SU-8 photosensitive resin was formed on a silicon wafer as shown in FIG. 2 to form a casting frame (Height 100 μm). Then, liquid PDMS was solidified in the casting frame to form a chip, and the chip was attached to a slide glass to form the microfluidic channel apparatus.

In the embodiment, an inlet of the microfluidic channel was divided into a sample inlet (one channel) and buffer solution inlets (eight channels), and an outlet was formed of eight channels. The sample inlet was disposed at a side surface to check behaviors of biological material samples coated with magnetic nanoparticles, and the number of buffer solution channels was increased to eight in order to reduce the parabolic fluid flow having the laminar flow effect. Also, the microfluidic channel structure was manufactured to promptly flow a PBS solution mixed with 10% of BSA therethrough after being manufactured in order to reduce the phenomenon in which the biological materials are stuck to surfaces of the microfluidic channel and fluid bubbles form.

### Example 2

### Synthesis of Magnetic Nanoparticles

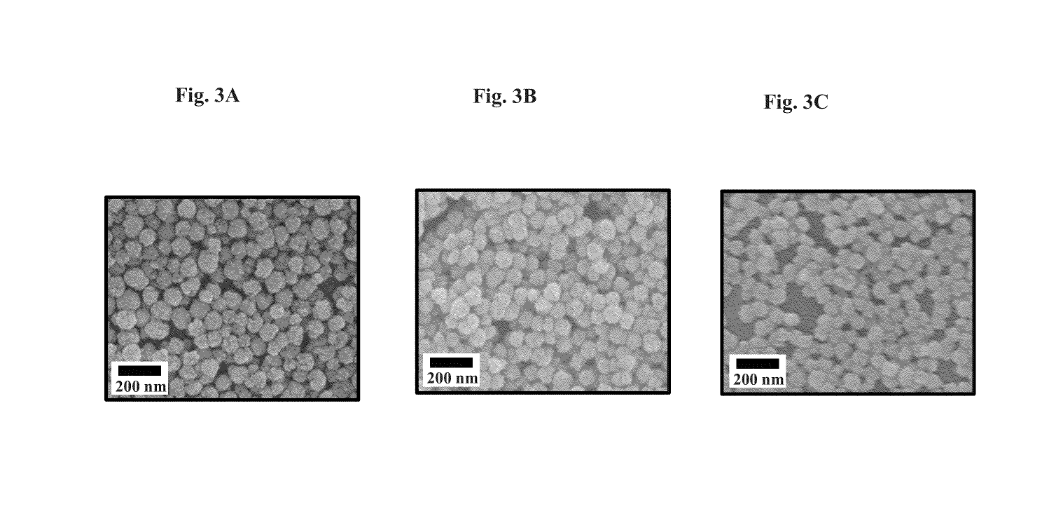

100 nm-magnetic nanoclusters were used as the magnetic nanoparticles. The magnetic nanoclusters were obtained by mixing FeCl₃, MCl₂ (M=Fe, Mn, or Co), sodium acetate, and polyacrylic acid (M_{ω}=1,800) in a mixture solution of diethylene glycol and ethylene glycol, and reacting for six hours or more in an electric furnace with solvothermal method. The composition of the magnetic nanoparticles was MFe₂O₄ (M=Fe, Mn, or Co), and the magnetic nanoparticles have surfaces shown in FIGS. 3A-3C and an average particle size of 100 nm. Magnetic nanoparticles of various sizes may be synthesized by controlling the amount of a sample.

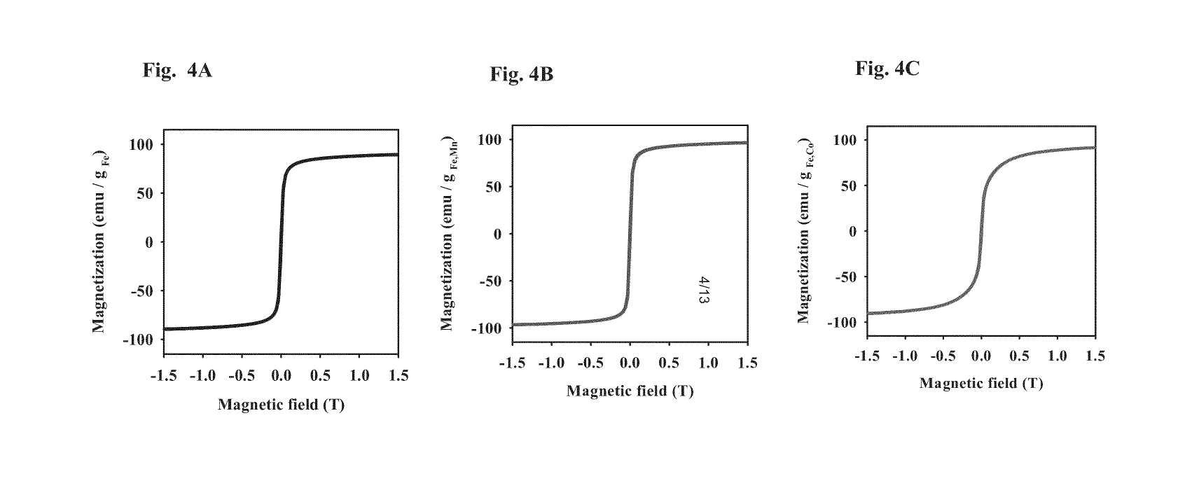

In FIGS. 4A-4C, magnetic susceptibility of each of the magnetic nanoparticles was measured to understand the characteristics. The separation of the micro-particles is possible using the differences in the magnetic susceptibilities.

### Example 3

### Fabrication Single Cell Coated with Magnetic Nanoparticle Cluster Having Different Compositions

After coating 10⁶ jurkat cells [ATCC, TIB-152, USA], which are human T lymphocyte cells, with 10 μg of three types of magnetic nanoparticles CoFe₂O₄, Fe₃O₄, MnFe₂O₄ having different compositions, the jurkat cells were cultured for 30 minutes, and the cells were fixed using paraformaldehyde. Then, the cells were treated with triton X to reduce cell aggregation.

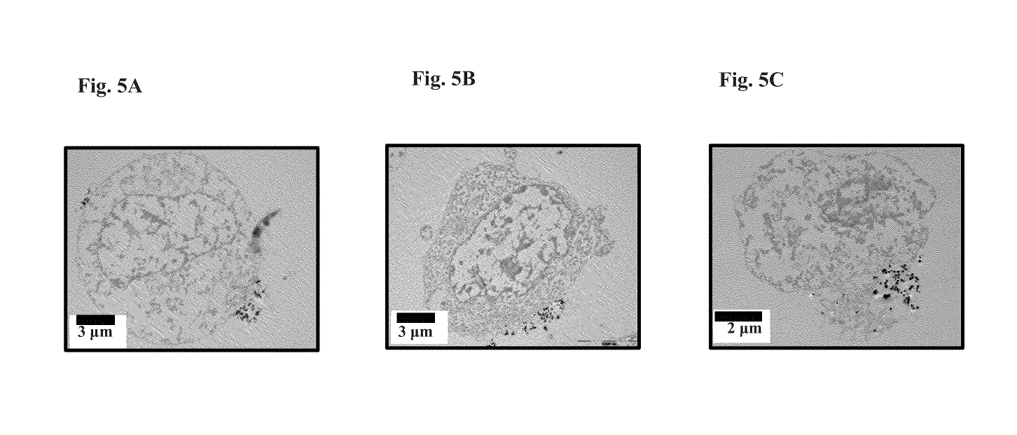

FIGS. 5A-5C illustrate electron microscopy images of the cells treated as above.

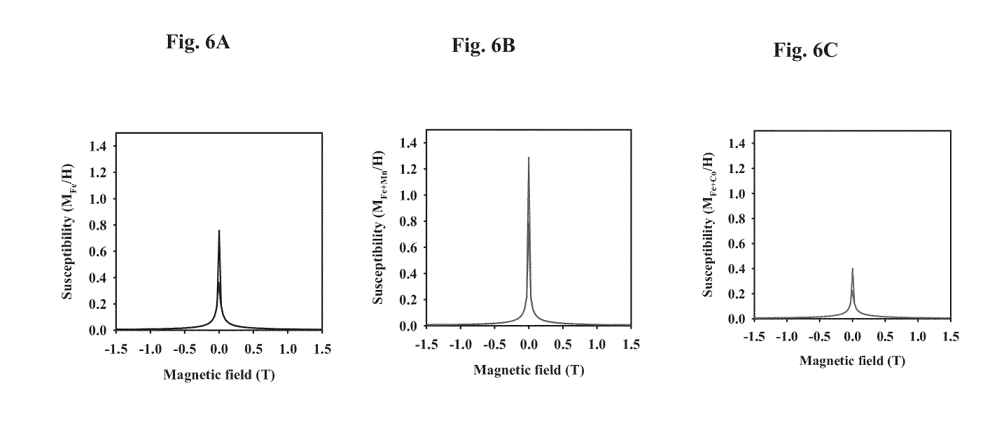

FIGS. 6A-6C illustrate results of measuring the magnetic susceptibility after treating the jurkat cells with each of the magnetic nanoparticles by the VSM [magnetic susceptibility of the jurkat cells treated with each of A) Fe₃O₄; B) MnFe₂O₄; C) CoFe₂O₄].

### Example 4

### Fabrication Cells Coated with Magnetic Nanoparticle Clusters Having Different Compositions

Antibodies [Anti-alpha 1 Sodium Potassium ATPase antibody [464.6], Plasma Membrane Marker, bought from abcam] coupled to ATPase surface antigens were used as jurkat cells [ATCC, TIB-152, USA] which are T-lymphocyte cell strains, antibodies [Anti-ErbB 2 antibody [3B5], bought from abcam] coupled to ERBB2 surface antibodies were used as SK-BR-3 cells [ATCC, HTB-30, USA] which are breast cancer cell strains, and antibodies [bought from Cetuximab, ImClone Systems Corporation] coupled to ERBBI surface antibodies were used for A431 cells [ATCC, CRL-1555, USA] which are epidermal cancer cell strains. For the antigen-antibody reactions, magnetic bodies with antibodies attached thereto were manufactured by amide coupling the antibodies corresponding to the antigens specifically expressed in each of the cell strains using 1-ethyl 3-dimethylaminopropyl carbodiimide and hydroxysuccinimide. Each of 10⁶ jurkat cells, SK-BR-3 cells, and A431 cells were coated with 10 μg of the magnetic nanoparticles (Fe₃O₄, MnFe₂O₄, CoFe₂O₄), and the cells were fixed using paraformaldehyde. Then, the cells were treated with triton X to reduce cell aggregation.

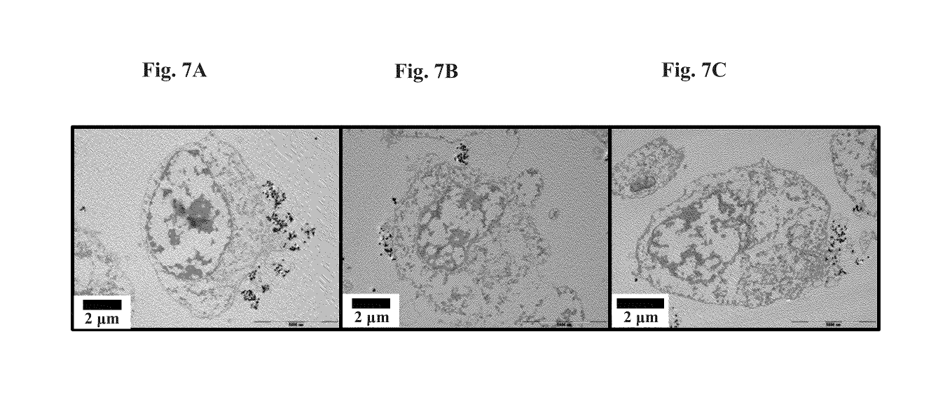

FIGS. 7A-7C illustrate electron microscopy images of the cells treated as above.

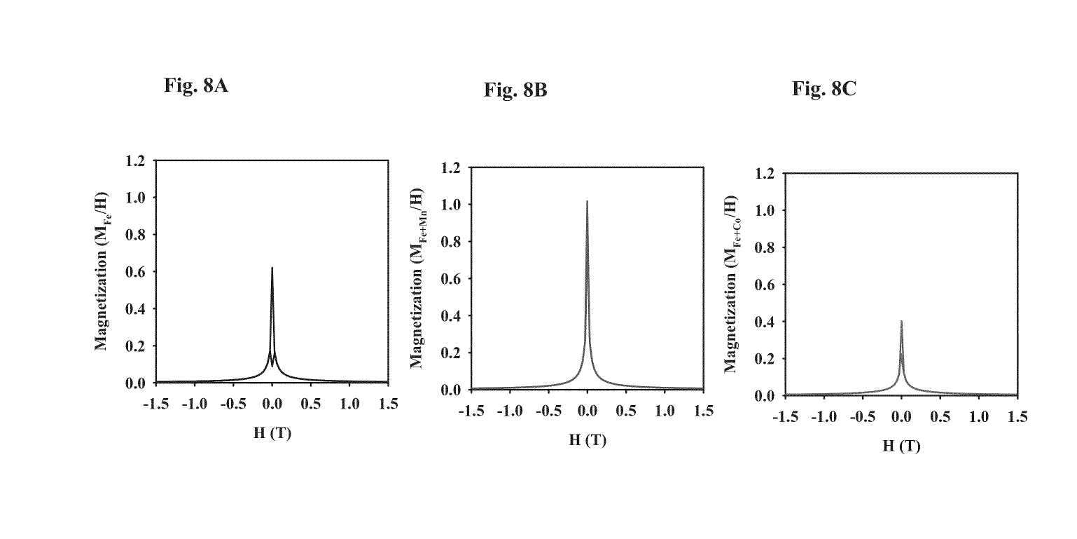

FIGS. 8A-8C illustrate results of measuring magnetic susceptibility after treating each of three types of cells with each of the magnetic nanoparticles by the VSM [magnetic susceptibility of A) the jurkat cells coated with Fe₃O₄; B) the SK-BR-3 cells coated with MnFe₂O₄; C) the A431 cells coated with CoFe₂O₄].

### Embodiment 1

### Trajectory of Cells Coated with Magnetic Nanoclusters in Microfluidic Channel

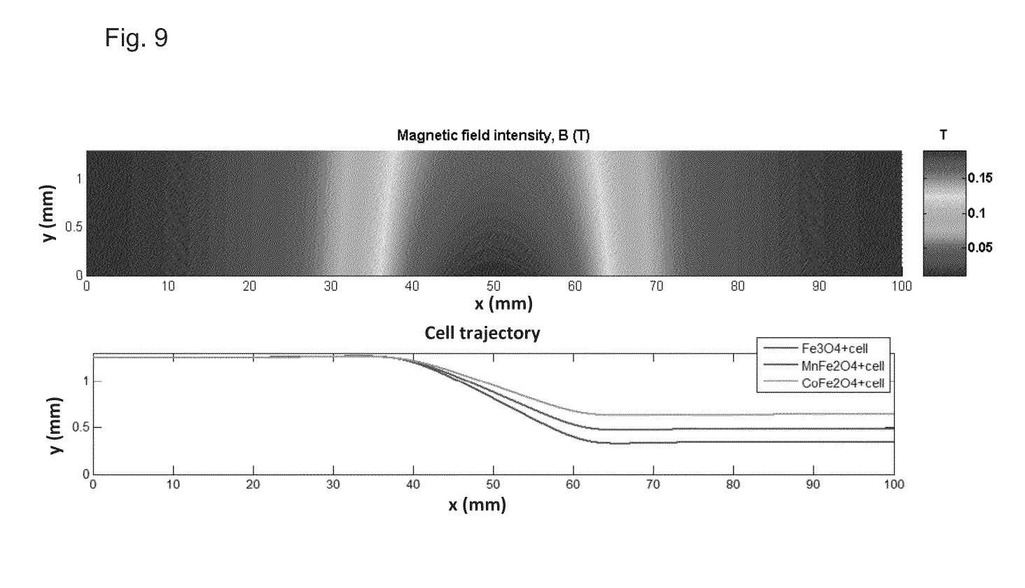

After simulating differences in cellular behavior by fixing a total flow amount of 90 μl/min (10 μl/min at the sample inlet, 80 μl/min at the buffer solution inlets) in the microfluidic channel with information of cells coated with the magnetic nanoparticles obtained from Example 3 above and controlling the size of the external magnetic field, results shown in FIG. 9 were obtained.

The magnetic field was made constant by putting the NdFeB magnet 5 mm away from the wall of the channel. It can be seen that a variance of changes in the behaviors of cells coated with the magnetic nanoparticle MnFe₂O₄, which had the greatest magnetic susceptibility, was the greatest, and a variance of changes in the behaviors of cells coated with the magnetic nanoparticle CoFe₂O₄, which had the smallest magnetic susceptibility, was the smallest.

### Embodiment 2

### Trajectory of Cells Coated with Magnetic Nanoclusters in Microfluidic Channel

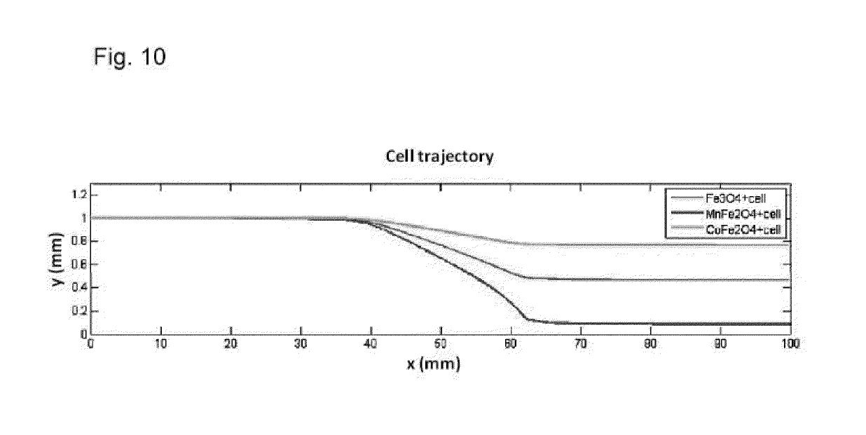

After simulating differences in cellular behavior by fixing a total flow amount of 90 μl/min (10 μl/min at the sample inlet, 80 μl/min at the buffer solution inlets) in the microfluidic channel with information of cells coated with the magnetic nanoparticles obtained from Example 4 above and controlling the size of the external magnetic field, results shown in FIG. 10 were obtained.

The magnetic field was made constant by putting the NdFeB magnet 5 mm away from the wall of the channel. It can be seen that a variance of changes in the behaviors of cells coated with the magnetic nanoparticle MnFe₂O₄, which had the greatest magnetic susceptibility, was the greatest, and a variance of changes in the behaviors of cells coated with the magnetic nanoparticle CoFe₂O₄, which had the smallest magnetic susceptibility, was the smallest.

### Embodiment 3

### Multicellular Separation

The cellular separation was performed by fixing the total flow amount of 90 μl/min (10 μl/min at the sample inlet, 80 μl/min at the buffer solution inlets) in the microfluidic channel with information of cells coated with the magnetic nanoparticles obtained from Example 4 above and using an external magnetic field having the average size of 0.15 T.

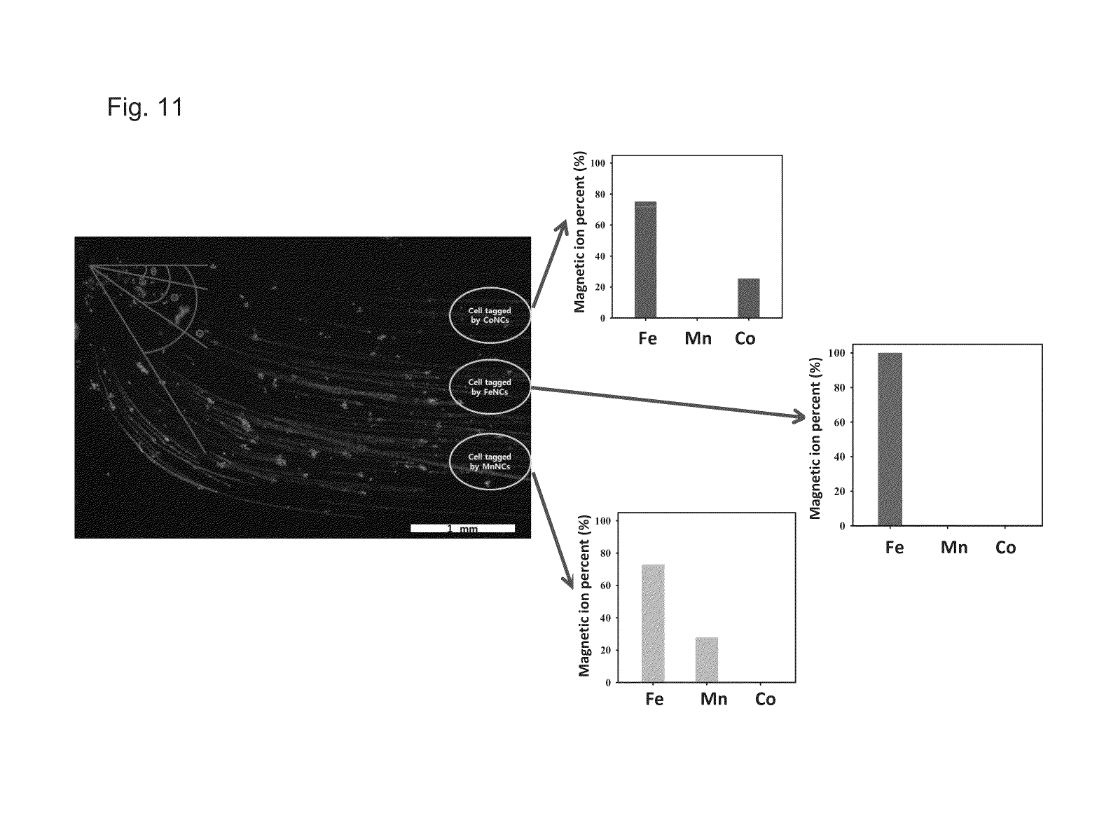

FIG. 11 illustrates a cellular image shown when each of the cells with the magnetic nanoparticles attached thereto was flowed through the microfluidic channel while having a magnet at a lower portion of the main channel of the microfluidic channel.

It can be seen that the susceptibility changes depending on the type of the certainly doped particle and causes a different degree of reacting to a magnetic force. Here, behaviors of the A431 cells coated with CoFe₂O₄, the jurkat cells coated with Fe₃O₄, and the SK-BR-3 cells coated with MnFe₂O₄ were respectively calculated as θ=11.3°, θ′=35.1°, and θ″=58.0°. Also, as a result of obtaining inductively coupled plasma data by spilling the cells in the microfluidic channel in a yellow circle portion and collecting the cells again, an elementary analysis result corresponding to the data of each portion was obtained.

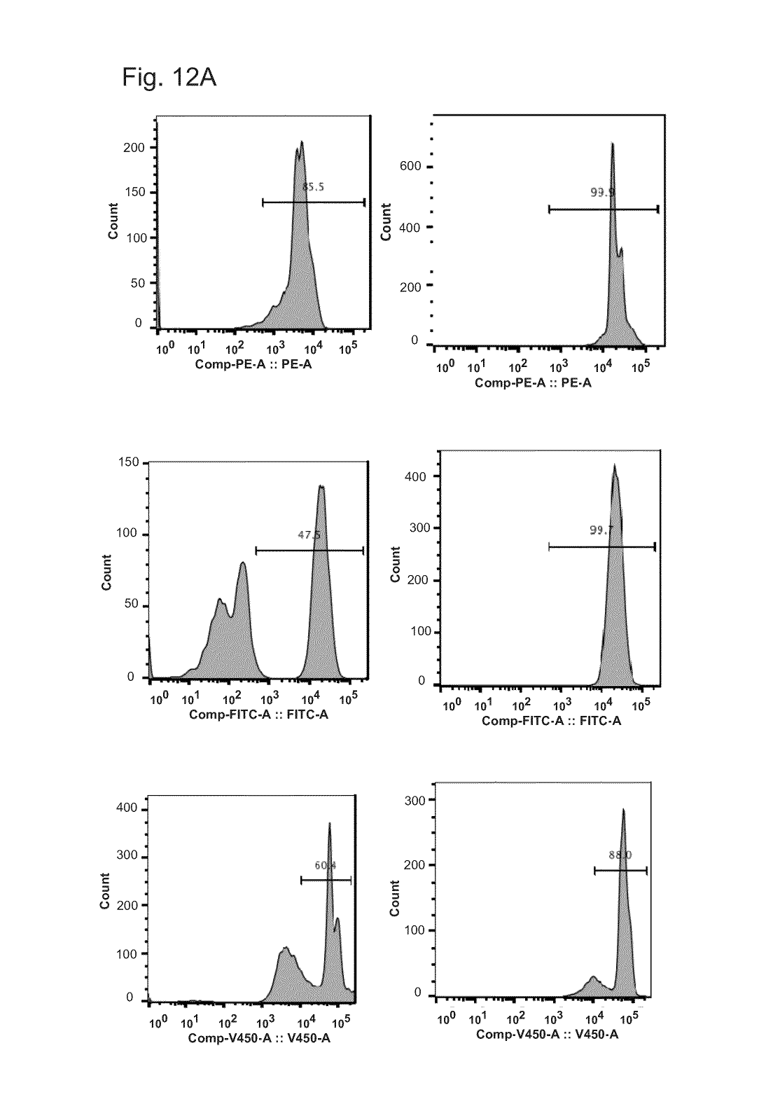

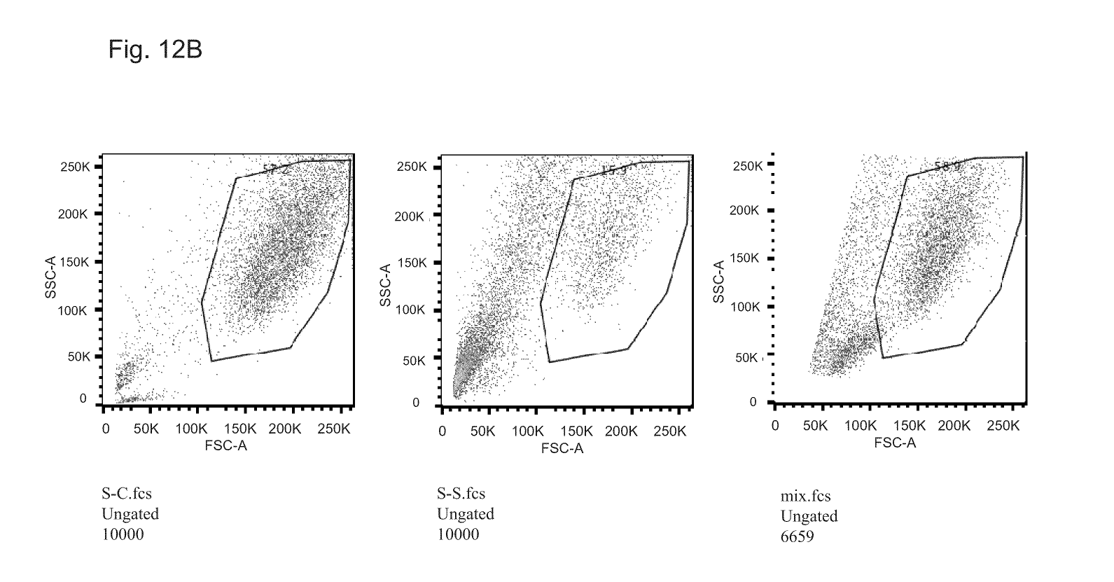

The mixture of the A431 cells coated with CoFe₂O₄, the jurkat cells coated with Fe₃O₄, and the SK-BR-3 cells coated with MnFe₂O₄ was injected into the injection unit of the microfluidic channel, the cells were separated by the same method as above, and the result was analyzed using FACS. As a result, as can be seen in FIG. 12A, the A431 cells were separated from 85.5% to 99.9% (first line of FIG. 12A), and the jurkat cells were separated from 47.5% to 99.7% (second line of FIG. 12A). The SK-BR-3 cells were separated from 60.4% to 88% (third line of FIG. 12A).

What is claimed is:

1. A method for separating multiple biological materials, the method comprising separating the multiple biological materials using magnetic susceptibility or magnetization of three or more types of magnetic nanoparticles having different compositions which are expressed by Chemical Formula 1 below:
MFe₂O₄ [Chemical Formula 1]
wherein M is Fe, Mn, Co, Ni, or Zn.

2. A method for separating multiple biological materials, the method comprising:
respectively coupling three or more types of magnetic nanoparticles to three or more types of biological materials to be separated in a sample;
injecting the sample and a buffer into a microfluidic channel;
generating a magnetic field outside of the microfluidic channel while the sample and the buffer are passing through the microfluidic channel; and
separating the biological materials to different movement pathways due to differences in magnetic susceptibility or magnetization of the magnetic nanoparticles,
wherein the magnetic nanoparticles are expressed by Chemical Formula 1 below:
MFe₂O₄ [Chemical Formula 1]
wherein M is Fe, Mn, Co, Ni, or Zn.

3. The method according to claim 1, wherein the biological materials may be viruses, bacteria, cells, intracellular organs, molecules, or multicellular organisms.

4. The method according to claim 1, wherein a size of the magnetic nanoparticles is 10 to 200 nm, and sizes of the magnetic nanoparticles having different compositions are the same.

5. The method according to claim 2, wherein the biological materials and the magnetic nanoparticles are coupled using an antigen-antibody reaction, a selective coupling reaction using an aptamer, or coupled using a surface charge.

6. The method according to claim 2, wherein an injection speed of the sample is 1 μl/min to 50 μl/min.

7. The method according to claim 2, wherein an injection speed of the buffer is 8 μl/min to 400 μl/min.

8. The method according to claim 2, wherein the magnetic field is generated in one direction different from a fluid flowing direction in the microfluidic channel.

9. The method according to claim 2, wherein an intensity of the magnetic field is 500 G to 3,000 G.

10. The method according to claim 1, using an apparatus for separating multiple biological materials, the apparatus comprising:
a microfluidic channel structure comprising an injection unit into which a plurality of samples and a buffer are injected, a main channel in which biological materials are separated by an external magnetic field, and a discharge unit configured to discharge a plurality of separated biological materials; and
a magnetic device configured to form a magnetic field along one direction different from a fluid flowing direction in the main channel.

11. The method according to claim 10, wherein, in the apparatus:
the injection unit comprises a sample inlet into which the samples are injected and buffer inlets into which the buffer is injected; and
the buffer inlets are formed of 8 to 20 channels.

12. The method according to claim 10, wherein, in the apparatus, the microfluidic channel structure is formed of a patterned polydimethyl siloxane channel on a lower glass substrate.

13. The method according to claim 10, wherein, in the apparatus, the magnetic device applies the external magnetic field from a permanent magnet or an electromagnet.

14. The method according to claim 2, wherein the biological materials may be viruses, bacteria, cells, intracellular organs, molecules, or multicellular organisms.

15. The method according to claim 2, wherein a size of the magnetic nanoparticles is 10 to 200 nm, and sizes of the magnetic nanoparticles having different compositions are the same.
